(19) United States
(12) Patent Application Publication
(10) Pub. No.: US 2014/0171516 A1
(43) Pub. Date: Jun. 19, 2014

(54) GERANYLGERANYLACETONE FORMULATIONS

(71) Applicant: COYOTE PHARMACEUTICALS, INC., MENLO PARK, CA (US)

(72) Inventor: Hiroaki Serizawa, Menlo Park, CA (US)

(21) Appl. No.: 13/819,681

(22) PCT Filed: Feb. 8, 2013
(86) PCT No.: PCT/US2013/025427

§ 371 Date: Feb. 27, 2013

Related U.S. Application Data
(60) Provisional application No. 61/605,155, filed on Feb. 29, 2012.

(51) Int. Cl. A61K 31/121

(52) U.S. Cl. CPC A61K 31/121
USPC 514/675; 435/375

(57) ABSTRACT

Provided herein is a pharmaceutical formulation comprising at least one geranylgeranyl acetone in the form of an eye drop. Also provided herein are methods of treating neural diseases or disorders by administering such pharmaceutical formulations.

### CROSS REFERENCE TO RELATED APPLICATION

This application is a national stage application under 35 U.S.C. §371 of International Application No. PCT/US13/25427, filed Feb. 8, 2013; which in turn claims priority under 35 U.S.C. section 119(e) of U.S. Provisional Application No. 61/605,155, filed on Feb. 29, 2012, which are incorporated herein by reference in their entireties.

### FIELD OF THE INVENTION

This invention relates to ocular formulations of geranylgeranyl acetone and methods of using them.

### STATE OF THE ART

It is difficult at best for an agent to penetrate into the eye and be delivered intraocularly. There is a need for delivering therapeutic agents into the eye, for example, for therapeutic purposes.

### SUMMARY OF THE INVENTION

This invention arises in part out of the surprising discovery that geranylgeranyl acetone (GGA) demonstrates highly effective intraocular penetration and administration into ocular tissue when administered on the ocular surface, or administered ocularly by topical delivery. The level achieved thereby into the ocular tissue was manifolds more, for example, about 5 times more than that achieved by systemic delivery by oral administration. For example, and without limitation, the levels of geranylgeranyl acetone achieved intraocularly by administering a topical ocular composition of 5% GGA was about 5 fold more than that achieved by administering 200 mg/kg GGA by systemic administration orally. Such is even more unexpected in view of the eye being a immunprotected organ which is predisposed to impede compounds from getting into the ocular tissue and to remove any compound entered into it.

Thus, according to certain preferred embodiments of this invention it is possible to administer a mixture of cis and all trans GGA and obtain a therapeutically effective concentration of trans GGA into the ocular tissue without showing potential negative effects of the cis GGA isomer. Such mixtures can contain, in some embodiments, about 30:70-40:60 ratio of the cis and the trans GGA isomers.

In some embodiments, the GGA is formulated as a thermosensitive gel. Thus formulated, a precursor sol is administered on the ocular surface where at an increased temperature, the sol undergoes a sol to gel transition. In some preferred embodiments, such gels comprise Polaxamers® as excipients. In some embodiments, the eye drop formulation forms a colored film once it contacts the ocular surface. Such a coloration allows an attending physician to determine the extent of the eye drop formulation retained on the ocular surface, and not spilled away from it, after delivery.

According to an aspect of this invention, a method is provided for inhibiting optic nerve damage in a patient at risk of such damage which method comprises applying a therapeutically effective amount of a composition comprising 0.0001 wt %-10 wt % geranylgeranyl acetone (GGA) to or into an ocular surface of said patient in an amount sufficient to increase intraocular levels of HSP 70, thereby inhibiting the optic nerve damage. In some preferred embodiments, the composition comprises 0.1 wt % to 10 wt % GGA. In other preferred embodiments, the composition comprises 3 wt % to 6 wt % GGA. In one embodiment, the invention provides a method for delivering unexpectedly high intraocular levels of GGA by administering GGA to an ocular surface of said patient.

According to yet another aspect of this invention, a method is provided for increasing HSP70 levels in ocular tissue comprising administering topically on the ocular surface an effective amount of geranylgeranyl acetone (GGA).

In some embodiments of this invention, the GGA is administered as a trans isomer free of the cis isomer or as a mixture of cis and trans isomers. In other embodiments of this invention, the method further includes providing an intraocular concentration of the GGA. In some preferred embodiments, the GGA is the all-trans isomer free of the cis isomer. In other preferred embodiments, the GGA is a mixture of cis and trans-isomers. In some embodiments of this invention, the intraocular levels of HSP 70 may be increased by at least 10%. In other embodiments of this invention, the optic nerve damage derives from or is related to glaucoma, macular degeneration, exposure to UV light, trauma, stroke, optic neuritis, ischemia, infection, compression from a tumor, compression from an aneurysm or Leber's hereditary optic neuropathy.

According to yet another aspect of this invention, a pharmaceutical composition is provided, where the pharmaceutical composition is suitable for parenteral administration through the ocular surface of a patient, wherein the pharmaceutical composition comprises geranylgeranyl acetone (GGA) and at least one excipient for introducing the GGA into the eye of a subject. In some embodiments of this invention, the pharmaceutical composition is suitable for parenteral administration through the ocular surface of a patient via a jetting device.

According to still another aspect of this invention, a pharmaceutical composition suitable for topical administration to a patient is provided, where the pharmaceutical composition comprises less than 0.01 wt % geranylgeranyl acetone (GGA) and at least one excipient for introducing the GGA into the eye of a subject, provided that the composition does not include an egg-based excipient, such as, for example, an egg-based phospholipid. Based on the surprising discoveries discussed herein, It is contemplated that even such small concentrations are suitable for administering a therapeutically effective amount of GGA, preferably into the eye.

Thus, in one embodiment, the invention provides pharmaceutical compositions suitable for topical administration that despite having low concentrations of GGA, deliver an effective concentration of GGA to a patient via the topical route. In certain preferred embodiments, the pharmaceutical composition comprises less than 0.005 wt % geranylgeranyl acetone (GGA). In other preferred embodiments, the pharmaceutical composition comprises less than 0.001 wt % geranylgeranyl acetone (GGA). In certain embodiments, the excipient for introducing the GGA into the eye of a subject comprises a tonicity adjustment agent.

In some preferred embodiments, the GGA is co-administered or administered in combination with beta-blockers and a steroid such as prostaglandin. Topical formulations, preferably ocular formulations, including GGA and one or more of a beta-blocker and a steroid, and uses thereof, preferably in treating optic nerve damage, such as those relating from glaucoma, are also contemplated according to this invention.

Provided herein, in some embodiments, is a topical ocular composition comprising (5E,9E,13E)geranylgeranyl acetone, wherein (5E,9E,13E)geranylgeranyl acetone is present in a ratio of greater than 90:10 of (5E,9E,13E) to (5Z,9E,13E)geranylgeranyl acetone isomers, and at least one tonicity adjusting agent. In some embodiments, the isotonic tonicity adjusting agent is isotonic. In specific embodiments, the tonicity adjusting agent is saline, dextrose, glycerin, aqueous potassium chloride, buffer salts, propylene glycol, or mannitol. In certain specific embodiments, the tonicity adjusting agent is saline. In some embodiments provided herein, the topical ocular composition is formulated as a topical eye drop. In some embodiments, the composition comprises about 0.1-5% of (5E,9E,13E) geranylgeranyl acetone. In some embodiments, the composition comprises about 0.1-2%, 0.1-1%, or 0.05-1% of (5E,9E,13E)geranylgeranyl acetone.

In some embodiments, the topical ocular composition further comprises one or more of a surfactant, an anti-bacterial agent, a pH buffering agent, an antioxidant agent, a preservative agent, a viscosity imparting agent or a combination thereof. In further or additional embodiments, the topical ocular composition is used for the manufacture of a medicament for the treatment of an ocular or visual disorder. In some embodiments, the ocular or visual disorder is a neurodegenerative disorder. In specific embodiments, the ocular or visual disorder is glaucoma, optic nerve degeneration or age-related macular degeneration.

Also provided herein in some embodiments is a physiological supplement or medicament for ophthalmic use, in the form of eye drops, comprising (5E,9E,13E)geranylgeranyl acetone in a range of about 0.5%-2.5%, wherein (5E,9E,13E)geranylgeranyl acetone is present in a ratio of greater than 90:10 of (5E,9E,13E) to (5Z,9E,13E)geranylgeranyl acetone isomers.

Some embodiments provided herein describe a formulation for treatment of an ocular neural disease, disorder or condition, comprising (5E,9E,13E)geranylgeranyl acetone, wherein (5E,9E,13E) geranylgeranyl acetone is present in a ratio of greater than 90:10 of (5E,9E,13E) to (5Z,9E,13E) geranylgeranyl acetone isomers, and at least one carrier material for introducing (5E,9E,13E) geranylgeranyl acetone into the eye of a subject suffering from the ocular neural disease, disorder or condition. In some embodiments, the formulation further comprises one or more of a surfactant, an anti-bacterial agent, a pH buffering agent, an antioxidant agent, a preservative agent, or a combination thereof. In some embodiments, the carrier material comprises an ocular/ophthalmic carrier. In some embodiments, the ocular neural disease, disorder, or condition is glaucoma, optic nerve degeneration or age-related macular degeneration.

Also provided herein in some embodiments is a method of treating glaucoma, the method comprising administering to a subject in need thereof a pharmaceutical formulation comprising (5E,9E,13E)geranylgeranyl acetone. In some embodiments, (5E,9E,13E)geranylgeranyl acetone is present in a ratio of greater than 90:10 of (5E,9E,13E) to (5Z,9E,13E)geranylgeranyl acetone isomers. In further or additional embodiments, the formulation further comprises one or more of a tonicity adjusting agent, a surfactant, an anti-bacterial agent, a pH buffering agent, an antioxidant agent, a preservative agent, a viscosity imparting agent or a combination thereof. In some embodiments, the formulation comprises 0.5-2.5% (5E,9E,13E)geranylgeranyl acetone. In some embodiments, the formulation is administered to the eye of the subject.

Some embodiments provided herein describe a method of inhibiting apoptosis of a retinal ganglion cell, the method comprising administration of a pharmaceutical formulation of (5E,9E,13E) geranylgeranyl acetone to the cell. In some embodiments, (5E,9E,13E)geranylgeranyl acetone is present in a ratio of greater than 90:10 of (5E,9E,13E) to (5Z,9E,13E)geranylgeranyl acetone isomers. In further or additional embodiments, the pharmaceutical formulation further comprises an ocular/ophthalmic carrier. In certain embodiments, the retinal ganglion cell is present in an individual. In some embodiments, the individual is in need of glaucoma therapy. In some embodiments, the pharmaceutical formulation is administered to the subject by an eye drop.

Provided herein in certain embodiments, is an eye drop for the treatment of an ocular neural disease, disorder or condition through topical application of said eye drop to the eye of a subject suffering from said disease, disorder or condition, comprising a therapeutically effective amount (5E,9E,13E) geranylgeranyl acetone and a solvent for said compound which is suitable for topical application to the eye of the subject, wherein (5E,9E,13E)geranylgeranyl acetone is present in a ratio of greater than 90:10 of (5E,9E,13E) to (5Z,9E,13E)geranylgeranyl acetone isomers.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### CERTAIN DEFINITIONS

Unless otherwise noted, terminology used herein should be given its normal meaning as understood by one of skill in the art.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “C₁-C₆-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₆-alkyl radicals include, but not limited to, methyl, ethyl, propyl, isopropyl, n-butyl, tert-butyl, neopentyl and n-hexyl.

The alkyl group may optionally be substituted by one or more of fluorine, chlorine, bromine, iodine, carboxyl, C₁₋₄ alkoxycarbonyl, C₁₋₄ alkylaminocarbonyl, di-(C₁₋₄ alkyl)-aminocarbonyl, hydroxyl, C₁₋₄ alkoxy, formyloxy, C₁₋₄ alkylcarbonyloxy, C₁₋₄ alkylthio, C₃₋₆ cycloalkyl or phenyl.

The term “aryl” as used herein, alone or in combination, refers to an optionally substituted aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “neuroprotective” refers to reduced toxicity of ocular neurons as measured, e.g., in vitro in assays where ocular neurons susceptible to degradation are protected against degradation as compared to control. Neuroprotective effects may also be evaluated in vivo by counting neurons in histology sections.

The term “neuron” or “neurons” refers to all electrically excitable cells that make up the ocular nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

As used herein, the term “protein aggregate inhibiting amount” refers to an amount of compound that inhibits the formation of protein aggregates at least partially or entirely. Unless specified, the inhibition could be directed to protein aggregates inside the cell or outside the cell.

As used herein, the term “intranuclear” or “intranuclearly” refers to the space inside the nuclear compartment of an animal cell.

The term “cytoplasm” refers to the space outside of the nucleus but within the outer cell wall of an animal cell.

As used herein, the term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

The term “ocular neurotransmitter” refers to chemicals which transmit signals from a neuron to a target cell in the eye.

The term “synapse” refers to junctions between ocular neurons. These junctions allow for the passage of chemical signals from one cell to another.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “carrier” as used herein, refers to relatively nontoxic chemical compounds or agents that facilitate the incorporation of a compound into cells or tissues.

The term “axon” refers to projections of neurons that conduct signals to other cells through synapses. The term “axon growth” refers to the extension of the axon projection via the growth cone at the tip of the axon.

The term “ocular neural disease” refers to diseases that compromise the cell viability of ocular neurons.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “cyclodextrin,” as used herein, refers to cyclic carbohydrates consisting of at least six to eight sugar molecules in a ring formation. The outer part of the ring contains water soluble groups; at the center of the ring is a relatively nonpolar cavity able to accommodate small molecules.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. An appropriate “effective” amount in any individual case may be determined using techniques, such as a dose escalation study.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The term “about” when used before a numerical designation, e.g., temperature, time, amount, and concentration, including range, indicates approximations which may vary by (+) or (−) 10%, 5%, or 1%.

The term “halogenating” is defined as converting a hydroxy group to a halo group. The term “halo” or “halo group” refers to fluoro, chloro, bromo and iodo.

The term “stereoselectively” is defined as providing over 90% of the one geometric isomer for a newly formed double bond.

“Geometrical isomer” or “geometrical isomers” refer to compounds that differ in the geometry of one or more olefinic centers. “E” or “(E)” refers to the trans orientation and “Z” or “(Z)” refers to the cis orientation.

Geranylgeranyl acetone (GGA) refers to a compound of the formula V:

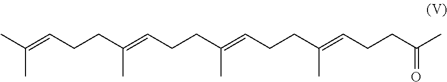 [SMILES: CC(=O)CC/C=C(\C)CC/C=C(\C)CC/C=C(\C)CCC=C(C)C]

wherein compositions comprising the compound are mixtures of geometrical isomers of the compound. The 5-trans isomer of geranylgeranyl acetone refers to a compound of the formula III:

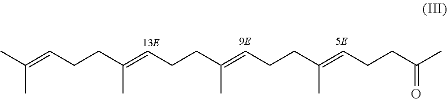 [SMILES: CC(=O)CC/C=C(\C)CC/C=C(\C)CC/C=C(\C)CCC=C(C)C]

wherein the number 5 carbon atom is in the 5-trans or 5E configuration. The 5-trans isomer also refers to (5E,9E,13E)geranylgeranyl acetone. The 5-cis isomer of geranylgeranyl acetone refers to a compound of the formula IV:

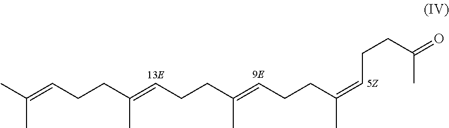 [SMILES: CC(=O)CC/C=C(/C)CC/C=C(\C)CC/C=C(\C)CCC=C(C)C]

wherein the number 5 carbon atom is in the 5-cis or 5Z configuration. The 5-cis isomer also refers to 5Z, 9E, 13E geranylgeranyl acetone. As used herein, geranylgeranyl acetone that is the all-trans isomer free of the cis isomer includes preferably less than 1%, more preferably less than 0.1%, or most preferably less than 0.01% of the cis-isomer.

### Compounds

Some embodiments of the present invention describe a pharmaceutical formulation comprising one or more isomers of a compound of formula I:

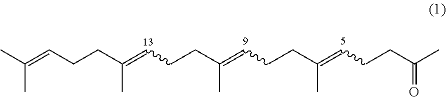 [SMILES: CC(=O)CCC=C(C)CCC=C(C)CCC=C(C)CCC=C(C)C]

in which the wavy line represents a bond having a configuration of the type (Z) or (E) or a mixture of the two configurations.

In some embodiments, geranylgeranyl acetone comprises a compound of formula II:

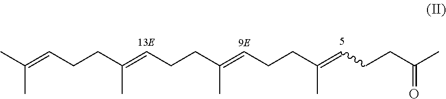 [SMILES: CC(=O)CCC=C(C)CC/C=C(\C)CC/C=C(\C)CCC=C(C)C]

in which the wavy line represents a bond having a configuration of the type (Z) or (E) or a mixture of the two configurations.

It will be clear to persons skilled in the art that in the compounds according to certain embodiments of the invention, the groups attached to the double bonds are fixed in different space as a result of the restricted rotation of double bonds. In some embodiments, provided herein is a compound of formula I or II, including all the stereoisomers, as well as mixtures thereof in any proportions, the Z and E isomers and mixtures thereof.

Preferably in the compounds of formula I or II according to certain embodiments of the invention, the 5-alkene has the E configuration. In certain specific embodiments, the compound of formula I or II is the 5-trans isomer of GGA. In some embodiments, a compound of Formula I or II has the (5E,9E,13E) configuration. In some embodiments, the compound of formula I or II has the formula III:

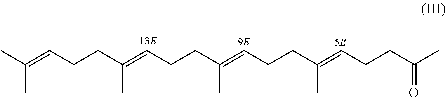 [SMILES: CC(=O)CC/C=C(\C)CC/C=C(\C)CC/C=C(\C)CCC=C(C)C]

In some embodiments, the compound of formula I, II or III is (5E,9E,13E)geranylgeranyl acetone. In some embodiments, the compound of formula I, II or III is in the form of a mixture of GGA isomers containing at least 80% by weight of the isomer having the (5E,9E,13E) configuration. In some embodiments, the compound of formula I, II or III is in the form of a mixture of GGA isomers containing at least 90% by weight of the isomer having the (5E,9E,13E) configuration. In some embodiments, the compound of formula I, II or III is in the form of a mixture of GGA isomers containing at least 85%, at least 90%, at least 95%, at least 96%, at least 97%, at least 98%, at least 99%, at least 99.5%, or at least 99.9% by weight of the isomer having the (5E,9E,13E) configuration. In other embodiments, the formulation comprises does not comprise a detectable amount of the 5-cis isomer of GGA. In other embodiments, the formulation comprises does not comprise a detectable amount of the GGA isomer of formula I or II having the 5Z, 9E, 13E configuration.

Other embodiments provided herein describe a pharmaceutical formulation comprising the 5-cis isomer of GGA. Some embodiments provided herein describe a pharmaceutical formulation comprising a compound of formula I or II wherein the 5-alkene has the Z configuration. In some embodiments, a compound of Formula I or II has the 5Z, 9E, 13E configuration. In some embodiments, the compound of formula I or II has the formula IV:

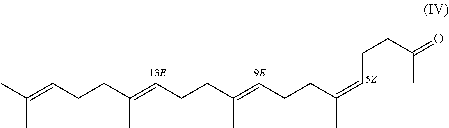 [SMILES: CC(=O)CC/C=C(/C)CC/C=C(\C)CC/C=C(\C)CCC=C(C)C]

In some embodiments, the compound of formula I, II or IV is (5E,9E,13E)geranylgeranyl acetone. In some embodiments, the compound of formula I, II, or IV in the form of a mixture of GGA isomers containing at least 80% by weight of the isomer having the (5E,9E,13E) configuration. In some embodiments, the compound of formula I, II or IV is in the form of a mixture of GGA isomers containing at least 80% by weight of the isomer having the 5Z, 9E, 13E configuration. In some embodiments, the compound of formula I, II or IV is in the form of a mixture of GGA isomers containing at least 85%, at least 90%, at least 95%, at least 96%, at least 97%, at least 98%, at least 99%, at least 99.5%, or at least 99.9% by weight of the isomer having the 5Z, 9E, 13E configuration. In some embodiments, the compound of formula I, II or IV is in the form of a mixture of GGA isomers containing at most 20%, at most 18%, at most 15%, at most 13%, at most 10%, at most 8%, at most 6%, at most 5%, at most 4%, at most 3%, at most 2%, at most 1%, or at most 0.5% by weight of the isomer having the 5Z, 9E, 13E configuration. In certain embodiments, the formulation comprises does not comprise a detectable amount of the 5-trans isomer of GGA. In other embodiments, the formulation comprises does not comprise a detectable amount of a compound of formula I, II or III having the (5E,9E,13E) configuration.

In some embodiments, any of the pharmaceutical formulations described herein comprise a compound of formula I, II, III, or IV, wherein the isomeric mixture of (5E,9E,13E) GGA to (5Z,9E, 13E) GGA is in a ratio of about 50:50, 60:40, 75:25, 80:20, 85:15, 90:10, 93:7, 95:5, 96:4, 97:3, 98:2, or 99:1. In some embodiments, (5E,9E,13E)geranylgeranyl acetone is present in a ratio of greater than 80:20 of (5E,9E,13E) to (5Z,9E,13E)geranylgeranyl acetone isomers. In some embodiments, (5E,9E, 13E)geranylgeranyl acetone is present in a ratio of greater than 85:15 of (5E,9E,13E) to (5Z,9E,13E) geranylgeranyl acetone isomers. In some embodiments, (5E,9E,13E)geranylgeranyl acetone is present in a ratio of greater than 90:10 of (5E,9E,13E) to (5Z,9E,13E)geranylgeranyl acetone isomers. In some embodiments, (5E,9E,13E)geranylgeranyl acetone is present in a ratio of greater than 95:5 of (5E,9E, 13E) to (5Z,9E,13E)geranylgeranyl acetone isomers. In some embodiments, (5E,9E,13E) geranylgeranyl acetone is present in a ratio of greater than 99:1 of (5E,9E,13E) to (5Z,9E,13E) geranylgeranyl acetone isomers.

The configuration of compounds is determined by methods known to those skilled in the art such as chiroptical spectroscopy and nuclear magnetic resonance spectroscopy.

A compound of formula I, II, III or IV may be synthesized according to the exemplary synthesis described below. For example, the compound of formula III is prepared following a method comprising one or more of the following steps:

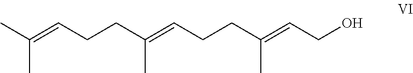 [SMILES: CC(C)=CCC/C(C)=C/CC/C(C)=C/CO]

(i) reacting a compound of formula VI under halogenation conditions to provide a compound of formula VII;

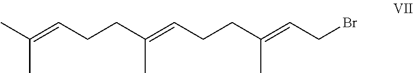 [SMILES: CC(C)=CCC/C(C)=C/CC/C(C)=C/CBr]

(ii) reacting the compound of formula VII with alkyl acetoacetate under alkylation conditions to provide a compound of formula VIII, where the stereochemistry at stereogenic center can be a racemic, R or S configuration:

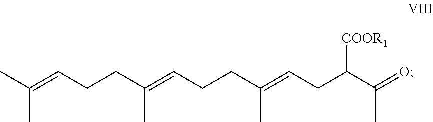

(iii) reacting the compound of formula VIII under hydrolysis and decarboxylation conditions to provide a compound of formula IX:

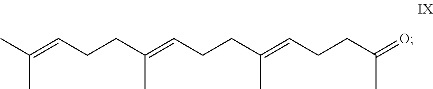 [SMILES: CC(=O)CC/C=C(\C)CC/C=C(\C)CCC=C(C)C]

(iv) reacting the compound of formula IX with a compound of formula X:

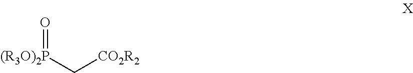

wherein R₂ and each R₃ independently are alkyl or substituted or unsubstituted aryl, under olefination conditions to selectively provide a compound of formula XI:

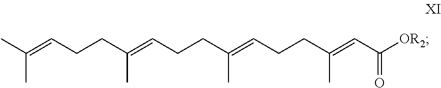

(v) reacting the compound of formula XI under reduction conditions to provide a compound of formula XII

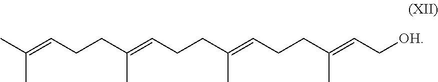 [SMILES: CC(C)=CCC/C(C)=C/CC/C(C)=C/CC/C(C)=C/CO]

Compound VI is combined with at least an equimolar amount of a halogenating agent typically in an inert solvent. As used in this application, an “inert solvent” is a solvent that does not react under the reaction conditions in which it is employed as a solvent. The reaction is typically run at a temperature of about 0° C. to 20° C. for a period of time sufficient to effect substantial completion of the reaction. Suitable solvents include, by way of example only, diethyl ether, acetonitrile, and the like. Suitable halogenating agents include PBr₃ or PPh₃/CBr₄. After reaction completion, the resulting product, compound IV, can be recovered under conventional conditions such as extraction, precipitation, filtration, chromatography, and the like or, alternatively, used in the next step of the reaction without purification and/or isolation.

Compound VII is combined with at least an equimolar amount of an alkyl acetoacetate, in the presence of a base and an inert solvent. The reaction is typically run initially at 0° C., and then warmed up to room temperature for a period of time sufficient to effect substantial completion of the reaction. Suitable solvents include, by way of example only, various alcohols, such as ethanol, dioxane, and mixtures thereof. Suitable bases include, by way of example only, alkali metal alkoxides, such as sodium ethoxide.

Compound VIII is reacted with at least an equimolar amount, preferably, an excess of aqueous alkali. The reaction is typically run at about 40 to 80° C. and preferably about 80° C. for a period of time sufficient to effect substantial completion of the reaction. Suitable solvents include, by way of examples only, alcohols, such as methanol, ethanol, and the like.

Compound IX is combined with at least an equimolar amount, preferably, an excess of a compound of formula X, and at least an equimolar amount, preferably, an excess of base, in an inert solvent. The reaction is typically run, initially at about −30° C. for about 1-2 hours, and at room temperature for a period of time sufficient to effect substantial completion of the reaction. Suitable solvents include, by way of examples only tetrahydrofuran, dioxane, and the like. Suitable bases include, by way of example only, alkali metal hydrides, such as sodium hydride, or potassium hexamethyldisilazide (KHMDS), or potassium tertiary butoxide (^{t}BuOK).

Compound XI is combined with a reducing agent in an inert solvent. The reaction is typically run at about 0° C. for about 15 minutes and at room temperature for a period of time sufficient to effect substantial completion of the reaction. Suitable reducing agents include, without limitation, LiAlH₄. Suitable solvents include, by way of examples only diethyl ether, tetrahydrofuran, dioxane, and the like.

As will be apparent to the skilled artisan, after reaction completion, the resulting product can be recovered under conventional conditions such as precipitation, filtration, chromatography, and the like or, alternatively, used in the next step of the reaction without purification and/or isolation.

In some embodiments, the method further comprises repeating steps (i), (ii), and (iii) sequentially with a compound of formula XII to provide a compound of formula V.

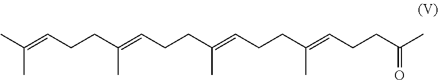 [SMILES: CC(=O)CC/C=C(\C)CC/C=C(\C)CC/C=C(\C)CCC=C(C)C]

In another embodiment, the synthetic method comprises repeating steps (i), (ii), (iii), (iv) and (v), sequentially, 1-3 times.

Also described herein is the synthetic method comprising one or more of the following steps:

(i) reacting a compound of formula XII:

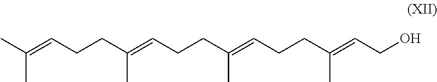 [SMILES: CC(C)=CCC/C(C)=C/CC/C(C)=C/CC/C(C)=C/CO]

under halogenation (e.g., bromination) condition to provide a compound of formula XIII

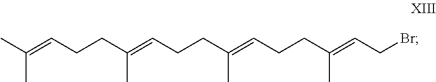 [SMILES: CC(C)=CCC/C(C)=C/CC/C(C)=C/CC/C(C)=C/CBr]

(ii) reacting the compound of formula XIII with alkyl acetoacetates, under alkylating conditions to provide a compound of formula XIV, where the stereochemistry at the stereogenic center is racemic or has an R or S configuration:

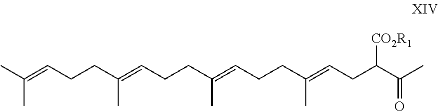

wherein R¹ alkyl is substituted or unsubstituted alkyl;

(iii) reacting a compound of formula XIV under hydrolysis and decarboxylation conditions to provide a compound of formula III:

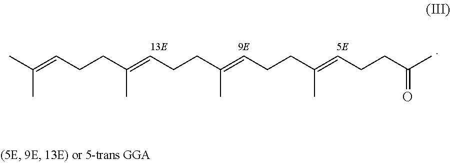 [SMILES: CC(=O)CC/C=C(\C)CC/C=C(\C)CC/C=C(\C)CCC=C(C)C]

An exemplary synthesis of the compound of formula IV is described herein, the method of synthesis comprising step (i) or step (ii) or steps (i) and (ii):

(i) reacting a compound of formula XV:

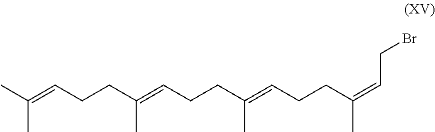 [SMILES: CC(C)=CCC/C(C)=C/CC/C(C)=C/CC/C(C)=C\CBr]

with alkyl acetoacetate under alkylating conditions to provide a compound of formula XVI, where the stereochemistry at the stereogenic center is racemic or has an R or S configuration:

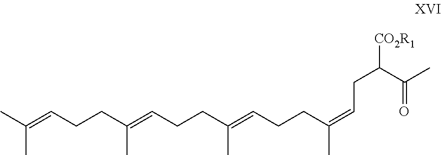

wherein R¹ alkyl is substituted or unsubstituted alkyl;

(ii) reacting a compound of formula XVI under hydrolysis and decarboxylation conditions to provide the compound of formula IV:

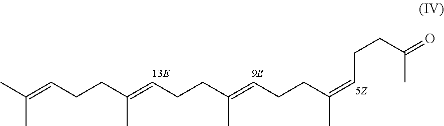 [SMILES: CC(=O)CC/C=C(/C)CC/C=C(\C)CC/C=C(\C)CCC=C(C)C]

In some embodiments, the compound of formula IV is synthesized by reacting a ketal compound of formula XVII:

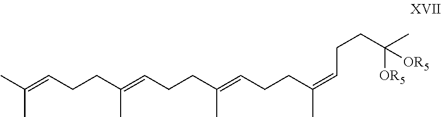

Wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups, under hydrolysis conditions to provide a compound of formula IV.

The ketal is combined with at least a catalytic amount, such as, 1-20 mol % of an aqueous acid, preferably, an aqueous mineral acid in an inert solvent. The reaction is typically run about 25° C. to about 80° C., for a period of time sufficient to effect substantial completion of the reaction. Suitable acids include, without limitation, HCl, H₂SO₄, and the like. Suitable solvents include alcohols, such as methanol, ethanol, tetrahydrofuran, and the like.

It will be apparent to the skilled artisan that the methods further employ routine steps of separation or purification to isolate the compounds, following methods such as chromatography (e.g., fractional distillation through a Fisher column), distillation (e.g., Kugelrohr distillation), or crystallization.

### Eye Drop Formulation

The compositions are formulated for eye delivery. Such formulations are well known in the art and can be modified based on this disclosure. As is well known, such formulations comprise sterile water and one or more excipients such as preservatives, antioxidants, tonicity adjusting agents, and the likes. IN some embodiments, the excepients further comprise, Polaxemers® and similar agents that can undergo a sol to gel transition upon delivery on the ocular surface. Alternatively, the compositions can be formulated for injection into the eye. Such are also well known.

Some embodiments provided herein describe a eye drop or ophthalmic formulation comprising a compound of formula I, II, III, IV, or (5E,9E,13E)geranylgeranyl acetone and an inert, non-eye irritating, non-toxic eye drop formulation. Such formulations are well known, and commonly referred to in, for example, the Physician's Desk Reference for Ophthalmology (1982 Edition, published by Medical Economics Company, Inc., Oridell, N.J.), wherein numerous sterile ophthalmologic ocular solutions are reported, e.g., see pp. 112-114, which are incorporated by reference.

Eye drop or ophthalmic formulations may include an excipient for introducing the GGA into the eye of a subject. Non-limiting examples of such an excipient for eye drop or ophthalmic formulations include a vehicle, tonicity adjusting agent, surfactant, stabilizer or anti-oxidant, viscosity imparting agent, acidic substance, preservative, diluent, wetting agent, and a buffering agent.

Reference is made herein to medicaments in the form of eye drops. In some embodiments, eye drops include solutions, suspensions, gels, creams and ointments intended for ophthalmic use. In some embodiments, the eye drops are applied with an eye dropper.

Some embodiments provided herein describe an eye drop formulation, wherein the concentration of a compound of formula I, II, III, IV, or (5E,9E,13E)geranylgeranyl acetone is about 0.0001-about 10 wt %, about 0.1-about 5 wt %, about 0.1-about 3 wt %, about 0.05-about 3 wt %, about 0.05-about 2 wt %, about 0.05-about 1 wt %, about 0.5-about 10 wt %, about 0.5-about 5 wt %, about 0.5-about 4 wt %, about 0.5-about 3 wt %, about 0.5-about 2 wt %, about 0.5-about 1 wt %, about 10%, about 7%, about 5%, about 4%, about 3.5%, about 3%, about 2.5%, about 2%, about 1.5%, about 1%, about 0.5%, about 0.1%, or about 0.05%. As is apparent and well known to the skilled artisan, the concentration of the active agent can be adjusted during and prior to the ocular delivery such that an effective amount is administered.

Some embodiments provided herein describe an eye drop formulation that comprises a vehicle. Examples of suitable vehicles for the eye drop formulation include but are not limited to purified water and vegetable oils (e.g., olive oil, castor oil, sesame oil, etc.).

Also provided herein in some embodiments is an eye drop formulation wherein the formulation further comprises one or more tonicity adjusting agents. In some embodiments, the tonicity adjusting agent is 0.5% to 2% of saline. In specific embodiments, the saline is a 0.9% w/v sodium chloride solution). Other non-limiting examples of tonicity adjusting agents include potassium chloride, buffer salts, dextrin, glycerin, propylene glycol and mannitol.

Some embodiments provided herein describe an eye drop formulation that optionally comprises a surfactant. In some embodiments, non-ionic surfactants aid in dispersing the active ingredient (e.g., (5E,9E, 13E)geranylgeranyl acetone) in suspensions and improve solution clarity. Non-limiting examples of suitable surfactants include sorbitan ether esters of oleic acid (e.g., polysorbate80 or Tween 20 and 80), polyoxyethylene hydrogenated castor oil, cremophor, sodium alkylbenzene sulfonate, glycerol, lecithin, sucrose ester, polyoxyethylene-alkyl ether, polyoxyl stearate, polyoxyl 40 stearate, polymers of oxyethylated octyl phenol (tyloxapol) and polyoxyethylene polyoxypropylene glycol. In some embodiments, the eye drop formulation comprises polysorbate80, polyoxyethylene hydrogenated castor oil, lecithin or combinations thereof. In some embodiments, the amount of surfactant is 0.2-30 times of (5E,9E,13E)geranylgeranyl acetone, but preferably 0.3-10 times of (5E,9E,13E)geranylgeranyl acetone. In some embodiments, an eye drop formulation comprises about 0.1-10 wt % of polysorbate80, polyoxyethylene hydrogenated castor oil, or lecithin. In some embodiments, an eye drop formulation comprises about 0.1-10 wt %, about 0.1-7 wt %, about 0.1-5 wt %, about 0.1-4 wt %, about 0.1-3 wt %, about 0.1-2 wt %, about 0.1-15 wt %, about 1-10 wt %, about 2-10 wt %, about 2-8 wt %, about 2-5 wt %, about 5-10 wt %, about 5-15 wt % of surfactant (e.g., polysorbate80, polyoxyethylene hydrogenated castor oil, or lecithin).

Some embodiments provided herein describe an eye drop formulation that optionally comprises a stabilizer or anti-oxidant. In some embodiments, the stabilizer or anti-oxidant decreases the rate of decomposition of active ingredient (e.g., (5E,9E,13E)geranylgeranyl acetone). Non-limiting examples of stabilizers and anti-oxidants include sodium bisulfate, sodium metabisulfite, ascorbic acid, isoascorbic acid, acetyl cysteine, 8-hydroxyquinoline, and thiourea.

Also provided herein in some embodiments is an eye drop formulation wherein the formulation further comprises one or more viscosity imparting agents. In some embodiments, viscosity imparting agents increase the viscosity of ophthalmic solution and suspension. In some embodiments, viscosity imparting agents increase ocular contact time, thereby decreasing the drainage rate. In some embodiments, viscosity imparting agents increase mucoadhesion, ocular bioavailability and/or impart a lubricating effect. Examples of viscosity imparting agents include but are not limited to poly vinyl alcohol, polyvinylpyrrolidone, methylcellulose, hydroxylpropylmethylcellulose, hydroxyethylcellulose, and carbomers.

In some embodiments, an acidic substance is optionally added. An example of an acidic substance is dimyristoylphosphatidic acid. Furthermore, adding dipalmitoylphosphatidylcholine (DPPG) results in more easily being able to prepare a clear solution. In some embodiments, anti-oxidants such as tocopherols or EDTA are added.

In some embodiments, preservatives are added to the eye drop formulation. In some embodiments, preservatives are anti-microbial or anti-bacterial agents. Parabens such as methylparaben and propylparaben, alcohol derivatives such as chlorobutanol, phenethyl alcohol, and benzyl alcohol, and organic acids such as sodium dehydroacetate, sorbic acid, and sodium sorbate are examples of such preservatives. Other examples of suitable preservatives include but are not limited to benzalkonium chloride, benzethonium chloride, polyquaternium-1 (Polyquad), thimerosal, phenylmercuric nitrate, phenylmercuric acetate, chlorobutanol, benzyl alcohol, sorbic acid, methyl paraben, propyl paraben, chlorhexidine, disodium EDTA, phenyl ethyl alcohol, polyaminopropyl biguanide, cetrimonium chloride, and purite. In some embodiments, the amount of preservative ranges from about 0.004% to about 0.02% by weight of the eye drop formulation.

Commonly used wetting agents are well known, and again are mentioned in the previously referred to pages of the Physician's Desk Reference for Ophthalmology. One suitable one is Tween, and in particular, Tween 80. In some embodiments, the amount of wetting agent ranges from 0.01% to 0.10%.

In some embodiments, the diluent is an isotonic eye treatment carrier, buffered to a pH within the range of from about 4.0 to about 8.0 and containing a small but effective amount of a wetting agent and an anti-bacterial agent.

Some embodiments provided herein describe an eye drop formulation optionally comprising one or more buffering agents. In some embodiments, the eye drops are buffered to about pH 7.4. In certain embodiments, the buffered eye drops maintain stability for at least 2 years. In some embodiments, the pH for the formulation described herein is within the range generally acceptable for eye drop, preferably pH 4-8 or about pH 7. The preferred pH range is from about 6.8 to about 7.8. Examples of suitable buffering agents include but are not limited to borate buffers and phosphate buffers (e.g., sodium phosphate).

For the manufacture of eye drop, a surfactant is added to a compound of formula I, II, III, IV, or (5E,9E,13E)geranylgeranyl acetone and mixed, and purified water is then added to the mixture. An isotonic agent such as sodium chloride and glycerin, buffer such as sodium phosphate, a pH-controlling agent such as dilute hydrochloric acid and sodium hydroxide, an antiseptic such as disodium edetate, an antifungal agent such as potassium sorbate, an anti-oxidizing agent such as tocophenol etc., is optionally added.

Eye drops are tested for various physicochemical, in vitro, and in vivo properties. Clarity is measured and ophthalmic solutions should be free from foreign particles. Visual and fluorescent microscopic methods are used for checking the clarity. The presence of particulate matter is also determined Light obscuration or microscopic methods are used for counting and or measuring the particle size. The light obscuration particle count test determines number of particles 50/mL (≧10 μm diameter) or 5/mL (≧25 μm diameter). The microscopic particle count test determines the number of particles 50/mL (≧10 μm diameter) or 5/mL (≧25 μm diameter) or 2/mL (≧50 μm).

Isotonicity of the formulation is tested. Isotonic solutions do not change shape (bulging or shrinkage) of blood cells. Any change in the shape of blood cells is compared with standard marketed formulation. pH meters are used to measure the pH of eye drops. Sedimentation time for particles in ophthalmic suspension is measured by visual and microscopical methods.

Ophthalmic suspensions are evaluated for resuspendability. The container is inverted at the rate of about 8-10 times in a minute, and the number of inversions required to completely re-suspend the settled particles is noted.

Drug content in ophthalmic formulation is evaluated by suitable analytical methods such as UV, HPLC.

Eye drops are tested for preservative effectiveness as per guidelines given in USP 30. The test recommends for screening the eye drops for the absence of E. coli, S. aureus, P. aeruginosa, C. albicans and A. niger.

Limulus amoebocyte lysate (LAL) test is used for determination of bacterial endotoxins. The test (pyrogen test) involves measuring the rise in temperature of rabbits following the intravenous injection of a test solution.

The formulation is also sterilized. Various sterilization methods are used to sterile the eye drops described herein, including steam sterilization, dry heat sterilization, gas sterilization, sterilization by ionizing radiation, sterilization by filtration, and aseptic processing.

### Methods of Treatment

Some embodiments provided herein describe a method of treating an ocular neural disease. In some instances, the ocular neural diseases are characterized by neuroinflammation. Also provided herein in some embodiments is a method of treating visual disorders such as optic neuropathy, glaucoma, degeneration of optic nerves, age-related macular degeneration (AMD) and ophthalmoplegia. Any pharmaceutical formulation and/or compounds described above are useful in the methods described herein.

Provided herein, in some embodiments, are methods for using effective amounts of one or more compounds of formula I, II, III or IV, preferably having the (5E,9E,13E) configuration or the, optionally with at least one pharmaceutically acceptable excipient for inhibiting ocular neural death and/or increasing neural activity. In some embodiments, the compound formula I, II, III or IV is the trans-GGA or the synthetic trans-GGA. For example, and without limitation, methods provided here in describe impeding the progression of ocular neural diseases or injury using one or more compounds of formula I, II, III or IV.

In one aspect, methods for increasing the axon growth of ocular neurons by contacting said neurons with the pharmaceutical compositions are provided herein. In some cases, ocular neural diseases result in an impairment of signaling between ocular neurons. In some cases, this impairment is due in part to a reduction in the growth of axonal projections. In some embodiments, contacting neurons with a compound of formula I, II, III, IV, or GGA enhances axonal growth. In some embodiments, a compound of formula I, II, III, IV, or GGA restores axonal grown in neurons afflicted with an ocular neural disease. In a related embodiment, the pre-contacted neurons exhibit a reduction in the axon growth ability.

One embodiment provided herein describes a method for inhibiting the cell death of ocular neurons susceptible to neuronal cell death, which method comprises contacting said neurons with the pharmaceutical compositions provided herein. Ocular neurons susceptible to neuronal cell death include those that have the characteristics of a neural disease and/or those that have undergone injury or toxic stress.

In another aspect, there are methods for increasing the ocular neurite growth of ocular neurons by contacting said neurons with the pharmaceutical compositions provided herein. The term “neurite” refers to both axons and dendrites. Ocular neural diseases can result in an impairment of signaling between ocular neurons. In some cases, this impairment is due in part to a reduction in the growth of axonal and/or dendritic projections. It is contemplated that contacting neurons with a compound of formula I, II, III, IV, or GGA will enhance ocular neurite growth. It is further contemplated that a compound of formula I, II, III, IV, or GGA will restore neurite grown in neurons afflicted with an ocular neural disease. In a related embodiment, the pre-contacted neurons exhibit a reduction in the neurite growth ability.

One embodiment of this invention is directed to a method for increasing the expression and/or release of one or more ocular neurotransmitters from an ocular neuron by contacting said neuron with the pharmaceutical compositions provided herein. It is contemplated that contacting ocular neurons with an effective amount of a compound of formula I, II, III, IV, or GGA will increase the expression level of one or more ocular neurotransmitters. It is also contemplated that contacting ocular neurons with a compound of formula I, II, III, IV, or GGA will increase the release of one or more ocular neurotransmitters from neurons. The release of one or more ocular neurotransmitters refers to the exocytotic process by which secretory vesicles containing one or more ocular neurotransmitters are fused to cell membrane, which directs the ocular neurotransmitters out of the neuron. It is contemplated that the increase in the expression and/or release of ocular neurotransmitters will lead to enhanced signaling in neurons, in which levels of expression or release of ocular neurotransmitters are otherwise reduced due to the disease. The increase in their expression and release can be measured by molecular techniques commonly known to one skilled in the art.

One embodiment of this invention is directed to a method for inducing synapse formation of an ocular neuron by contacting said neuron with the pharmaceutical compositions provided herein. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount of a compound of formula I, II, III, IV, or GGA will increase synapse formation in an ocular neurons that otherwise experience reduced synapse formation as a result of neural disease.

Another embodiment of this invention is directed to a method for increasing electrical excitability of an ocular neuron by contacting said neuron with the pharmaceutical compositions provided herein. Electrical excitation is one mode of communication among two or more neurons. It is contemplated that contacting neurons with an effective amount of a compound of formula I, II, III, IV, or GGA will increase the electrical excitability of ocular neurons in which electrical excitability and other modes of neural communication are otherwise impaired due to neural disease. Electrical excitability can be measured by electrophysiological methods commonly known to one skilled in the art.

In another embodiment, this invention is directed to a method for inhibiting the death of ocular neurons due to formation of or further formation of pathogenic protein aggregates between, outside or inside neurons, wherein said method comprises contacting said neurons at risk of developing said pathogenic protein aggregates with the pharmaceutical compositions provided herein. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell.

Another embodiment of the invention is directed to a method for protecting ocular neurons from pathogenic extracellular protein aggregates which method comprises contacting said neurons and/or said pathogenic protein aggregates with the pharmaceutical compositions provided herein. In one embodiment of this invention, contacting said neurons and/or said pathogenic protein aggregates with the pharmaceutical compositions provided herein. There are many assays known to one skilled in the art for measuring the protection of neurons either in cell culture or in a mammal.

In yet another embodiment of the invention is directed to a method for protecting ocular neurons from pathogenic intracellular protein aggregates which method comprises contacting said neurons with the pharmaceutical compositions provided herein.

One embodiment of the invention is directed to a method of modulating the activity of G proteins in ocular neurons which method comprises contacting said neurons with the pharmaceutical compositions provided herein. It is contemplated that contacting neurons with a compound of formula I, II, III, IV, or GGA will alter the sub-cellular localization, thus changing the activities of the G protein in the cell. In one embodiment of the invention, contacting neurons with a compound of formula I, II, III, IV, or GGA will enhance the activity of G proteins in ocular neurons. It is contemplated that contacting a compound of formula I, II, III, IV, or GGA with neurons will increase the expression level of G proteins. It is also contemplated that contacting a compound of formula I, II, III, IV, or GGA with optical neurons will enhance the activity of G proteins by changing their sub-cellular localization to the cell membranes where they must be to exert their biological activities.

One embodiment of the invention is directed to a method of modulating or enhancing the activity of G proteins in ocular neurons at risk of death which method comprises contacting said neurons with the pharmaceutical compositions provided herein.

One embodiment of the invention is directed to a method for inhibiting ocular neural death and increasing ocular neural activity in a mammal suffering from ocular neural diseases, wherein the etiology of said neural diseases comprises formation of protein aggregates which are pathogenic to ocular neurons, and which method comprises administering to said mammal the pharmaceutical compositions provided herein. This method is not intended to inhibit ocular neural death and increase ocular neural activity in ocular neural diseases in which the pathogenic protein aggregates are intranuclear or diseases in which the protein aggregation is related to SBMA.

In some embodiments, a pharmaceutical formulation described herein exerts cytoprotective effects on the eye. (See, for example Ishii Y., et al., Invest Ophthalmol Vis Sci 2003; 44:198292; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 20; 104:1837-43; and Suzuki S, et al., Kidney Int 2005; 67:2210-20).

Some embodiments provided herein describe methods for treating eye-related diseases, disorders or conditions with a compound of formula I, II, III, IV, or GGA. Examples of eye-related or visual disorders include but are not limited to macular degeneration, retinitis pigmentosa, glaucoma, and/or retinal degeneration.

In some embodiments, a pharmaceutical formulation described herein comprising a compound of formula I, II, III, IV or GGA is used for treating glaucoma. Glaucoma is a degenerative disease of the eye characterized by progressive optic nerve damage with selective loss of retinal ganglion cells. In some instances, apoptosis leads to retinal ganglion cell death in glaucoma. In some instances, the intraocular pressure remains elevated for prolonged time periods, the fibers of the optic nerve atrophy and/or the retina loses function.

Accordingly, provided herein is a method of inhibiting apoptosis-like cell death of retinal ganglion cells comprising administering to the retinal ganglion cell a pharmaceutical formulation comprising a compound of formula I, II, III, IV or (5E,9E,13E) GGA. In some embodiments, a method is provided for enhancing the survival of retinal ganglion cells. In further or additional embodiments, a method is described protecting retinal ganglion cells from damage or cell death. Also provided herein in some embodiments is a method for inducing expressing of heat shock proteins (e.g., HSP72) in a retinal neuron. In some embodiments, a method of ameloriating glaucomatous damage to an eye comprises administration of a pharmaceutical formulation comprising a compound of formula I, II, III, IV or (5E,9E, 13E) GGA. In other embodiments, a method is provided for preventing axonal injury in an optic nerve, the method comprising administering to the eye a pharmaceutical formulation comprising a compound of formula I, II, III, IV or (5E,9E,13E) GGA. Some embodiments provided herein describe a method of reducing elevated intraocular pressure in an eye comprising administering to the eye a pharmaceutical formulation comprising a compound of formula I, II, III, IV or (5E,9E,13E) GGA. In specific embodiments, the pharmaceutical formulation is administered to the eye as a drop, spray or ointment.

In certain aspects, the methods described herein relate to administering a compound of formula I, II, III, IV, or GGA or the isomeric compounds or compositions thereof in vitro. In other aspects the administration is in vivo. In yet other aspects, the in vivo administration is to a mammal. Mammals include but are not limited to humans and common laboratory research animals such as, for example, mice, rats, dogs, pigs, cats, and rabbits.

Compounds, compositions and methods of the invention described herein include the disclosures found in international application No.: PCT/US2011/050071, filed on Aug. 31, 2011 and the international PCT application entitled “GERANYLGERANYLACETONE DERIVATIVES”, filed on Feb. 29, 2012, both of which are incorporated herein in its entirety by reference. All citations herein are incorporated herein by reference in their entirety.

### Examples

### Example 1

### Eye Drop Formulation of (5E,9E,13E) Geranylgeranyl Acetone

Eye drops are prepared by dissolving (5E,9E,13E)geranylgeranyl acetone (1.0 g) in a phosphate buffer solution which is prepared by dissolving 0.8 g of sodium dihydrogen phosphate and 0.5 g of sodium chloride in purified water such that the final weight is 100 g. The pH was adjusted to 7.0 with sodium hydroxide.

### Example 2

### Eye Drop Formulation

Eye drops are prepared by dissolving (5E,9E,13E)geranylgeranyl acetone (1.0 g) in 1.0 g of dimethyl sulfoxide and adding the resulting solution to a boric acid solution prepared by dissolving 2.0 g of boric acid in purified water such that the final weight is 100 g. The pH was adjusted to 7.0 with sodium hydroxide.

### Example 3

### Eye Drop Formulation

| (5E, 9E, 13E) geranylgeranyl acetone | 1.0 | g |
|---|---|---|
| Potassium sorbate | 0.1 | g |
| Polysorbate80 | 0.5 | g |
| Sodium chloride | 0.9 | g |
| Disodium edetate | 0.01 | g |
| Sodium hydroxide | as appropriate | |
| Dilute hydrochloric acid | as appropriate | |
| Total Volume | 100 | mL |

Polysorbate80 is added to (5E,9E,13E)geranylgeranyl acetone in sterile purified water. After mixing, potassium sorbate, sodium chloride, and disodium edetate in sterile purified water is added to the mixture and stirred. The pH is adjusted to 6.5 by adding sodium hydroxide in sterile purified water and dilute hydrochloric acid.

### Example 4

### Eye Drop Formulation

The eye drop formulation (in 100 mL) is prepared following similar methods described in Example 3.

| | | |
|---|---|---|
| (5E, 9E, 13E) geranylgeranyl acetone | 1.0 | g |
| Potassium sorbate | 0.2 | g |
| Polysorbate80 | 0.5 | g |
| Sodium chloride | 0.81 | g |
| Disodium edetate | 0.01 | g |

| | |
|---|---|
| Sodium hydroxide | as appropriate |
| Dilute hydrochloric acid | as appropriate |

### Example 5

### Eye Drop Formulation

The eye drop formulation (in 100 mL) is prepared following similar methods described in Example 3.

| | |
|---|---|
| (5E, 9E, 13E) geranylgeranyl acetone | 0.5 g |
| Potassium sorbate | 0.2 g |
| Polysorbate80 | 0.25 g |
| Sodium chloride | 0.81 g |
| Disodium edetate | 0.01 g |
| Sodium hydroxide | as appropriate |
| Dilute hydrochloric acid | as appropriate |

### Example 6

### Eye Drop Formulation

The eye drop formulation (in 100 mL) is prepared following similar methods described in Example 3.

| | |
|---|---|
| (5E, 9E, 13E) geranylgeranyl acetone | 0.2 g |
| Potassium sorbate | 0.5 g |
| Polyoxyethylene hydrogenated castor oil | 2.0 g |
| Sodium chloride | 0.8 g |
| Disodium edetate | 0.01 g |
| Sodium hydroxide | as appropriate |
| Dilute hydrochloric acid | as appropriate |

### Example 7

### Eye Drop Formulation

The eye drop formulation (in 100 mL) is prepared following similar methods described in Example 3.

| | |
|---|---|
| (5E, 9E, 13E) geranylgeranyl acetone | 5.0 g |
| Potassium sorbate | 1.0 g |
| Polyoxyethylene hydrogenated castor oil | 2.5 g |
| Sodium chloride | 0.8 g |
| Disodium edetate | 0.05 g |
| Sodium hydroxide | as appropriate |
| Dilute hydrochloric acid | as appropriate |

### Example 8

### Eye Drop Formulation

| (5E, 9E, 13E) geranylgeranyl acetone | 100 | mg |
|---|---|---|
| Egg yolk lecithin | 50 | mg |
| DMPA (dimyristoylphosphatidic acid) | 10 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

The eye drop in this invention is manufactured in the following fashion. After dissolving (5E,9E, 13E)geranylgeranyl acetone, egg yolk lecithin (the phospholipid), and tocopherol acetate in a solvent mixture of chloroform and methanol, the solvent is distilled off using an evaporator, leaving a thin film of lipids. 5% glucose solution is added and shaken to suspend the lipids, then exposed to ultrasound, for example 15 minutes in a 40° C. ultrasonic bath. A synthetic surfactant, Tween 80 solution for example, is added, and then more 5% glucose solution is added to produce a clear (5E,9E,13E)geranylgeranyl acetone-containing eye drop.

### Example 9

### Eye Drop Formulation

| (5E, 9E, 13E) geranylgeranyl acetone | 100 | mg |
|---|---|---|
| Egg yolk lecithin | 35 | mg |
| DMPA | 7 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 10

### Eye Drop Formulation

| (5E, 9E, 13E) geranylgeranyl acetone | 100 | mg |
|---|---|---|
| Egg yolk lecithin | 15 | mg |
| DMPA | 3 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 11

### Eye Drop Formulation

| (5E, 9E, 13E) geranylgeranyl acetone | 100 | mg |
|---|---|---|
| Egg yolk lecithin | 0 | mg |
| DMPA | 0 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 12

### Eye Drop Formulation

| (5E, 9E, 13E) geranylgeranyl acetone | 100 | mg |
|---|---|---|
| Vitamin E | 1 | mg |
| Egg yolk lecithin | 50-100 | mg |
| DMPA | 0-12 | mg |
| Cholesterol | 0-16 | |
| Tween 80 | 50 | mg |
| Glycerin | 1-2 | mg |
| Potassium sorbate | 20 | mg |
| Britton-Robinson buffer | 0-1 | mL |
| 0.3M boric acid buffer pH 9 | 0-1 | mL |
| EDTA-2Na | 0-0.4 | mg |
| Sodium hydroxide in water | as appropriate | |
| Sterile water | as appropriate | |
| Total volume | 10 | mL |

### Example 13

### Eye Drop Formulation of (5E,9E,13E) Geranylgeranyl Acetone

Physiological saline and (5E,9E,13E)geranylgeranyl acetone is dropped (one drop each in the eyes of 10 persons) to thus inspect the preparations for the feeling (ocular irritation) observed during the period ranging from the time immediately after the application thereof to 3 minutes after the application.

### Example 14

### Permeability Study with Eye Drop Formulation

An ophthalmic solution is made up as follows: 1 mg/ml (0.1%) solution of (5E,9E,13E) geranylgeranyl acetone in phosphate buffered saline (pH=7.4) is used for half of the experiments and 1 mg/ml (0.1%) solution of (5E,9E,13E)geranylgeranyl acetone in phosphate buffered acrylic acid suspension is used for the experiments on rabbit corneas.

Before each permeability experiment, rabbit cornea tissue specimens are thawed at room temperature in phosphate buffered saline (PBS, pH 7.4). Tissue disks are equilibrated for 10 minutes with PBS (pH 7.4) at 20° C. in both the donor and receiver compartments of the diffusion cells.

Following equilibration, the PBS is removed from the donor compartment and replaced with 1.0 mL of PBS, containing 1 mg/mL (0.1%) (5E,9E,13E)geranylgeranyl acetone in PBS at pH 7.4 (w/v). PBS at 20° C. is pumped through the receiving chambers at a rate of 1.5 mL/h with a ISMATEC® 16 Channel High precision tubing pump and collected, by means of a ISCO Retriever IV fraction collector, at 2 h intervals for 24 h. The permeability studies are performed under sink conditions, i.e., at the completion of each run the concentration of (5E,9E,13E)geranylgeranyl acetone solution in the acceptor chamber never reaches 10% of that in the donor compartment. (5E,9E,13E)geranylgeranyl acetone containing samples are collected in appropriate sampling tubes of the fraction collector. Samples are analyzed by HPLC with UV detection. The collected fractions were analyzed directly after completion of the respective experiment for (5E,9E,13E)geranylgeranyl acetone content.

Calculation of Flux Values: Flux (J) values across membranes are calculated by means of the relationship J=Q/A×t (ng×cm-²×min⁻¹) where Q indicates quantity of substance crossing membrane (in ng); A, membrane area exposed (in cm²); and t, time of exposure (in minutes).

Steady State Kinetics: when no statistically significant differences (p<0.05; analysis of variance and Duncan's multiple range test) between flux values are obtained over at least two consecutive time intervals, a steady state (equilibrium kinetics) is assumed to have been reached for a particular corneal specimen.

### Example 15

### Eye Drop Formulation and In Vivo Study

Eye drops are made by dissolving sufficient quantity of (5E,9E,13E)geranylgeranyl acetone in distilled water to give 0.1%, 0.5%, 0.75%, and 2.0% solutions of (5E,9E,13E)geranylgeranyl acetone. Two drops are administered to the eye of normal and ocular induced hypertensive rabbits. The intraocular pressure of both the normal and ocular induced hypertensive rabbits is measured at intervals over a 6-hour period.

### Example 16

### Ocular Irritation Test

Rabbits are used as experimental animals (Draize test) for the measurement of redness, swelling, discharge, ulceration, hemorrhaging, cloudiness, or blindness in the tested eye. Confocal laser scanning ophthalmoscopy (CLSO) combined with corneal flourescein staining are also used.

### Example 17

### Rat Ocular Pharmacokinetics and Pharmacodynamics Study of Cis-Trans Geranylgeranyl Acetone and all-Trans Geranylgeranyl Acetone

Objective:

The objective of this study was to establish initial pharmacokinetic (PK) and pharmacodynamic (PD) data for an eye drop formulation containing geranylgeranyl acetone (GGA). In Cohort 1 the pharmacokinetics of all-trans geranylgeranyl acetone CNS-102 (“Formulation 102”) and cis-trans geranylgeranyl acetone CNS-101 (“Formulation 101”) were measured at different time points after multiple dose administrations. In Cohort 2 the efficacy of Formulation 102 was tested against Formulation 101 and vehicle controls at different time points.

Experimental Design:

Cohort 1: PK Study

One eye per rat was treated with geranylgeranyl acetone and one eye per rat was dosed with vehicle control according to the schedule shown in Table 1.

Cohort 2: HSP70 Analysis for ELISA Only

One eye per rat was treated with geranylgeranyl acetone and one eye per rat was dosed with vehicle control according to the schedule shown in Table 2.

TABLE 1
Dosing Schedule for the PK study

| | | Treatment | | | | |
|---|---|---|---|---|---|---|
| Group | # of | Dose Level | Dose Level | Dose | | Time of eye |
| # | Rats | Left Eye | Right eye | Volume | Dosing times | harvest |
| 1a | 3M | CNS-102 | 0 mg/eye | 5 μL | 0 h, 1 h, 2 h, 3 h | 4 h |
| | | 0.25 mg/eye | | | | |
| 2a | 3M | CNS-102 | 0 mg/eye | 5 μL | 0 h, 1 h, 2 h, 3 h, | 8 h |
| | | 0.25 mg/eye | | | 4 h, 5 h, 6 h, 7 h | |
| 3a | 3M | CNS-101 | 0 mg/eye | 5 μL | 0 h, 1 h, 2 h, 3 h | 4 h |
| | | 0.25 mg/eye | | | | |
| 4a | 3M | CNS-101 | 0 mg/eye | 5 μL | 0 h, 1 h, 2 h, 3 h, | 8 h |
| | | 0.25 mg/eye | | | 4 h, 5 h, 6 h, 7 h | |

TABLE 2
Dosing Schedule for the HSP70 analysis

| | | Treatment | | | | |
|---|---|---|---|---|---|---|
| Group | # of | Dose Level | Dose Level | Dose | | Time of eye |
| # | Rats | Left Eye | Right eye | Volume | Dosing times | harvest |
| 1b | 4M | CNS-102 | 0 mg/eye | 5 μL | 0 h, 1 h, 2 h, 3 h | 4 h |
| | | 0.25 mg/eye | | | | |
| 2b | 4M | CNS-102 | 0 mg/eye | 5 μL | 0 h, 1 h, 2 h, 3 h, | 8 h |
| | | 0.25 mg/eye | | | 4 h, 5 h, 6 h, 7 h | |
| 3b | 4M | CNS-101 | 0 mg/eye | 5 μL | 0 h, 1 h, 2 h, 3 h | 4 h |
| | | 0.25 mg/eye | | | | |
| 4b | 4M | CNS-101 | 0 mg/eye | 5 μL | 0 h, 1 h, 2 h, 3 h, | 8 h |
| | | 0.25 mg/eye | | | 4 h, 5 h, 6 h, 7 h | |
| 5b | 2M | Vehicle Ctrl | Vehicle Ctrl | 5 μL | 0 h, 1 h, 2 h, 3 h, | 8 h |
| | | 0 mg/eye | 0 mg/eye | | 4 h, 5 h, 6 h, 7 h | |

Dose Administration:

Route: topical eye drop formulation
Frequency: 4 or 8 doses, every 1 hour
Dose Administration: under isofluorane anesthesia (2.5%)
Dose Volume: 5 μL in each eye

### Formulation 102:

5% all-trans Geranylgeranyl acetone CNS-102 (oily liquid, clear, stored at −20° C.)
2.5% Hydrogenated castor oil
1% Potassium sorbate

### 0.8% NaCl

### 0.05% Disodium Edate

### In H₂O

pH 6.5

### Formulation 101:

5% cis-trans Geranylgeranyl acetone CNS-101
2.5% Hydrogenated castor oil
1% Potassium sorbate

0.8% NaCl

### 0.05% Disodium Edate

### In H₂O

pH 6.5

### Vehicle Control:

2.5% Hydrogenated castor oil
1% Potassium sorbate

### 0.8% NaCl

### 0.05% Disodium Edate

### In H₂O

pH 6.5

### Test Subjects:

### Species: Rat

### Strain: Sprague-Dawley

### Supplier: Harlan

### Sex: Male

### Weight at Initiation: 200 to 220 g

### Number of Animals: 12 for Cohort 1, 26 for Cohort 2

Further data is provided in Tables 3-9 below.

TABLE 3

| | | | Ratio |
|---|---|---|---|
| Animal | | | treated eye/vehicle |
| & eye | Treatment | HSP70 pg/ml | treated eye |
| #31L | CNS-102 | 583.588 | 1.259783888 |
| #31R | vehicle | 463.2445 | |
| #32L | CNS-102 | 701.7893 | 1.263196648 |
| #32R | vehicle | 555.5661 | |
| #33L | CNS-102 | 685.1953 | 0.595323064 |
| #33R | vehicle | 1150.964 | |
| #34L | CNS-102 | 757.1621 | 1.238701424 |
| #34R | vehicle | 611.2547 | |
| #35L | CNS-101 | 589.4561 | 1.003339056 |
| #35R | vehicle | 587.4945 | |
| #36L | CNS-101 | 1023.901 | 2.135270806 |
| #36R | vehicle | 479.5182 | |
| #37L | CNS-101 | 1211.234 | 1.54376015 |
| #37R | vehicle | 784.5996 | |
| #38L | CNS-101 | 985.2114 | 1.975865135 |
| #38R | vehicle | 498.6228 | |

TABLE 4
Ratio: compound treated/vehicle treated

| | CNS 102 | CNS 101 |
|---|---|---|
| Average (AVE) | 1.089251 | 1.664559 |
| Standard deviation (STDEV) | 0.329464 | 0.506717 |

TABLE 5
Left (compound treated)

| | | |
|---|---|---|
| AVE | 681.9337 | 952.4506 |
| STDEV | 72.42484 | 261.3504 |

TABLE 6
Right (vehicle treated)

| | | |
|---|---|---|
| AVE | 695.2573 | 587.5588 |
| STDEV | 309.8754 | 139.5319 |

TABLE 7

| Sample | | Dilution | Concentration | Mean of | |
|---|---|---|---|---|---|
| No. | Eye | Factor | (ng/g) | Duplicate (ng/g) | R/L |
| 1 | L | 1 | 21506 | | |
| 1 | L | 1 | 22150 | 21828 | 0.203729 |
| 2 | L | 1 | 17494 | | |
| 2 | L | 1 | 17194 | 17344 | 0.32703 |
| 3 | L | 2 | 32854 | | |
| 3 | L | 2 | 27764 | 30309 | 0.38482 |
| 4 | L | 1 | 14635 | | |
| 4 | L | 1 | 14809 | 14722 | 0.435708 |
| 5 | L | 1 | 15206 | | |
| 5 | L | 1 | 15120 | 15163 | 0.221262 |
| 6 | L | 1 | 19608 | | |
| 6 | L | 1 | 20376 | 19992 | 0.390131 |
| 7 | L | 1 | 12094 | | |
| 7 | L | 1 | 9398 | 10746 | 0.114368 |
| 8 | L | 1 | 8505 | | |
| 8 | L | 1 | 8285 | 8395 | 0.226742 |
| 9 | L | 1 | 7547 | | |
| 9 | L | 1 | 6790 | 7168.5 | 0.443956 |
| 10 | L | 1 | 18947 | | |
| 10 | L | 1 | 18145 | 18546 | 0.199558 |
| 11 | L | 1 | 12879 | | |
| 11 | L | 1 | 13519 | 13199 | 0.120918 |
| 12 | L | 1 | 18953 | | |
| 12 | L | 1 | 18638 | 18795.5 | 0.194887 |
| 1 | R | 1 | 4474 | | |
| 1 | R | 1 | 4420 | 4447 | |
| 2 | R | 1 | 5567 | | |
| 2 | R | 1 | 5777 | 5672 | |
| 3 | R | 1 | 11109 | | |
| 3 | R | 1 | 12218 | 11663.5 | |
| 4 | R | 1 | 6473 | | |
| 4 | R | 1 | 6356 | 6414.5 | |
| 5 | R | 1 | 3312 | | |
| 5 | R | 1 | 3398 | 3355 | |
| 6 | R | 1 | 7144 | | |
| 6 | R | 1 | 8455 | 7799.5 | |
| 7 | R | 1 | 1202 | | |
| 7 | R | 1 | 1256 | 1229 | |
| 8 | R | 1 | 1837 | | |
| 8 | R | 1 | 1970 | 1903.5 | |
| 9 | R | 1 | 3306 | | |
| 9 | R | 1 | 3059 | 3182.5 | |
| 10 | R | 1 | 3739 | | |
| 10 | R | 1 | 3663 | 3701 | |
| 11 | R | 1 | 1561 | | |
| 11 | R | 1 | 1631 | 1596 | |
| 12 | R | 1 | 3615 | | |
| 12 | R | 1 | 3711 | 3663 | |

TABLE 8

| | 4 x CNS-102 | | | 4 x CNS-101 | | |
|---|---|---|---|---|---|---|
| | 4 h ispi | 4 h contra | contra/ipsi | 4 h ispi | 4 h contra | contra/ipsi |
| AVE | 23160.33 | 7260.833 | 0.305193 | 16625.67 | 5856.333 | 0.349034 |
| STDEV | 6584.386 | 3861.704 | 0.092499 | 2923.657 | 2274.216 | 0.112976 |

TABLE 9

| | 8 x CNS-102 | | | 8 x CNS-101 | | |
|---|---|---|---|---|---|---|
| | 8 h ispi | 8 h contra | contra/ipsi | 8 h ispi | 8 h contra | contra/ipsi |
| AVE | 8769.833 | 2105 | 0.261689 | 16846.83 | 2986.667 | 0.171788 |
| STDEV | 1817.966 | 992.2158 | 0.16755 | 3161.578 | 1204.503 | 0.044116 |

### Example 18

### 1. HSP70 Induction after in Eyes by Eye Drops

Male Sprague-Dawley rats were administered an eye drop formulation containing 5% GGA. Eye drops were applied every hour either for 4 hours or for 8 hours. Animals were euthanized 4 hours, 8 hours or 24 hours after the first dosing, and the eye balls collected on ice. Eyes were homogenized with a polytron homogenizer in a standard lysis buffer containing proteinase inhibitors. HSP70 was quantified by a commercially available ELISA kit and normalized by total protein concentration in the sample.

### 2. PK after Administering Eye Drops

Male Sprague-Dawley rats were administered an eye drop formulation containing 5% GGA. Eye drops were applied every hour either for 4 hours or for 8 hours. Animals were euthanized 4 hours and 8 hours after the first dosing, and the eye balls collected on dry ice. Eyes were homogenized with a polytron homogenizer in ethanol. GGA was quantified in the eye ball lysates by liquid chromatography-tandem mass spectroscopy.

TABLE 10
HSP70 expression in eye balls following topical ocular administration
of 5% CNS-101

| | Vehicle | | | |
|---|---|---|---|---|
| | Control | 4 hours | 8 hours | 24 hours |
| HSP70 | 42.9 ± 6.79 | 50.7 ± 8.01 | 55.9 ± 11.2 | 50.5 ± 10.8 |
| [pg/mg | | | | |
| protein] | | | | |

mean ± SD

TABLE 11
CNS-101 concentrations in eye balls following topical ocular
administration of 5% CNS-101 measured after 4 hrs and 8 hrs,
respectively

| | Ocular |
|---|---|
| | Administration |
| | 4 × 5 ml 5% CNS-101 |
| CNS-101 | 16,600 ± 2,920 |
| [ng/g] | |
| | 8 × 5 ml 5% CNS-101 |
| CNS-101 | 16,800 ± 3,160 |
| [ng/g] | |

mean ± SD

### Example 19

### 1. PK Studies

Single dose of 5% GGA is administered by eye drop to rat eye balls (both eyes). 4-5 time points including time 0 are taken, as is base line data. AUC (eye ball) is calculated. A percentage of an input delivered to eye balls is calculated.

### 2. HSP70 Inductions

Single dose of 5% GGA is administered by eye drop to rat eye ball (both eyes). Eye balls are extracted at 2-3 time points. It is contemplated that HSP70 inductions in eye balls may be seen at different time points. Vehicle only controls using different animals are used. HSP70 induction in tissues dosed with GGA or vehicle is determined

### Example 20

Parenteral Administration of Geranylgeranyl Acetone Through the Ocular Surface of a Patient contemplated that a jetting device such as that described, e.g., and without limitation, in U.S. Pat. No. 7,563,244 can be used to administer an effective amount of geranylgeranyl acetone into the eye of a patient through the ocular surface of the patient. For example, a geranylgeranyl acetone formulation, such as Formulations 101 or 102, can be added to a jetting device that dispenses the formulation into the eye by ejecting it as a vapor or as droplets towards the ocular surface of the patient, whereby the pharmaceutical formulation penetrates the ocular surface and deliver geranylgeranyl acetone into the eye of a patient.

1. A method for inhibiting optic nerve damage in a patient at risk of such damage which method comprises applying a therapeutically effective amount of a composition comprising 0.0001-10 wt % geranylgeranyl acetone (GGA) to or into an ocular surface of said patient in an amount sufficient to increase intraocular levels of HSP 70, thereby inhibiting the optic nerve damage.

2. A method of increasing HSP70 levels in ocular tissue comprising administering topically on the ocular surface an effective amount of geranylgeranyl acetone (GGA).

3. The method of claim 2, wherein the GGA is administered as a trans isomer free of the cis isomer or as a mixture of cis and trans isomers.

4. The method of claim 1, further comprising providing an intraocular concentration of the GGA.

5. The method of claim 1, wherein the composition comprises 0.1 wt % to 10 wt % GGA.

6. The method of claim 1, wherein the composition comprises 3 wt % to 6 wt % GGA.

7. The method of claim 1, wherein the GGA is the all-trans isomer free of the cis isomer.

8. The method of claim 1, wherein the GGA is a mixture of cis and trans-isomers.

9. The method of claim 1, wherein the intraocular levels of HSP 70 are increased by at least 10%.

10. The method of claim 1, wherein the optic nerve damage derives from or is related to glaucoma, macular degeneration, exposure to UV light, trauma, stroke, optic neuritis, ischemia, infection, compression from a tumor, compression from an aneurysm or Leber's hereditary optic neuropathy.

11. A pharmaceutical composition suitable for parenteral administration to a patient, wherein the pharmaceutical composition comprises geranylgeranyl acetone (GGA) and at least one excipient for introducing the GGA into the eye of a subject.

12. The pharmaceutical composition of claim 11, suitable for parenteral administration through the ocular surface of a patient via a jetting device.

13. A pharmaceutical composition suitable for topical administration to a patient, wherein the pharmaceutical composition comprises less than 0.01 wt % geranylgeranyl acetone (GGA) and at least one excipient for introducing the GGA into the eye of a subject, provided that the composition does not include an egg-based excipient.

14. The pharmaceutical composition of claim 13, wherein the composition comprises less than 0.005 wt % geranylgeranyl acetone (GGA).

15. The pharmaceutical composition of claim 11, wherein the excipient for introducing the GGA into the eye of a subject comprises a tonicity adjustment agent.

16. A topical ocular composition comprising (5E,9E,13E)geranylgeranyl acetone, wherein (5E,9E,13E)geranylgeranyl acetone is present in a ratio of greater than 90:10 of (5E,9E,13E) to (5Z,9E,13E)geranylgeranyl acetone isomers, and at least one tonicity adjusting agent.

17. The topical ocular composition of claim 16, wherein the tonicity adjusting agent is isotonic.

18. The topical ocular composition of claim 16, wherein the tonicity adjusting agent is saline, dextrose, glycerin, aqueous potassium chloride, buffer salts, propylene glycol, or mannitol.

19. The topical ocular composition of claim 16, wherein the tonicity adjusting agent is saline.

20. The topical ocular composition of claim 16 in the form of a topical eye drop.

21. The topical ocular composition of claim 16, comprising 0.1-5% of (5E,9E,13E)geranylgeranyl acetone.

22. The topical ocular composition of claim 16, wherein the composition further comprises one or more of a surfactant, an anti-bacterial agent, a pH buffering agent, an antioxidant agent, a preservative agent, a viscosity imparting agent or a combination thereof.

23. The topical ocular composition of claim 16 for use in the manufacture of a medicament for treatment of an ocular or visual disorder.

24. The topical ocular composition of claim 23, wherein the ocular or visual disorder is a neural disorder.

25. The topical ocular composition of claim 24, wherein the neural disorder is glaucoma, optic nerve degeneration or age-related macular degeneration.

26. A physiological supplement or medicament for ophthalmic use, in the form of eye drops, comprising (5E,9E,13E)geranylgeranyl acetone in a range of about 0.5%-2.5%, wherein (5E,9E,13E)geranylgeranyl acetone is present in a ratio of greater than 90:10 of (5E,9E,13E) to (5Z,9E,13E)geranylgeranyl acetone isomers.

27. A formulation for treatment of an ocular neural disease, disorder or condition, comprising (5E,9E,13E)geranylgeranyl acetone, wherein (5E,9E,13E) geranylgeranyl acetone is present in a ratio of greater than 90:10 of (5E,9E,13E) to (5Z,9E, 13E)geranylgeranyl acetone isomers, and at least one carrier material for introducing (5E,9E, 13E)geranylgeranyl acetone into the eye of a subject suffering from the neural disease, disorder or condition.

28. The formulation of claim 27, further comprising one or more of a surfactant, an anti-bacterial agent, a pH buffering agent, an antioxidant agent, a preservative agent, or a combination thereof.

29. The formulation of claim 27, wherein said carrier material comprises an ocular/ophthalmic carrier.

30. The formulation of claim 27, wherein the neural disease, disorder, or condition is glaucoma, optic nerve degeneration or age-related macular degeneration.

31. A method of treating glaucoma, the method comprising administering to a subject in need thereof a pharmaceutical formulation comprising (5E,9E,13E)geranylgeranyl acetone.

32. The method of claim 31, wherein (5E,9E,13E)geranylgeranyl acetone is present in a ratio of greater than 90:10 of (5E,9E,13E) to (5Z,9E,13E)geranylgeranyl acetone isomers.

33. The method of claim 31, wherein the formulation further comprises one or more of a tonicity adjusting agent, a surfactant, an anti-bacterial agent, a pH buffering agent, an antioxidant agent, a preservative agent, a viscosity imparting agent or a combination thereof.

34. The method of claim 31, wherein the formulation comprises 0.5-2.5% (5E,9E,13E)geranylgeranyl acetone.

35. The method of claim 31, wherein the formulation is administered to the eye of the subject.

36. A method of inhibiting apoptosis of a retinal ganglion cell, the method comprising administration a pharmaceutical formulation of (5E,9E,13E)geranylgeranyl acetone to the cell.

37. The method of claim 36, wherein (5E,9E,13E)geranylgeranyl acetone is present in a ratio of greater than 90:10 of (5E,9E,13E) to (5Z,9E,13E)geranylgeranyl acetone isomers.

38. The method of claim 36, wherein the pharmaceutical formulation further comprises an ocular/ophthalmic carrier.

39. The method of claim 36, wherein the retinal ganglion cell is present in an individual.

40. The method of claim 36, wherein the individual is in need of glaucoma therapy.

41. The method of claim 36, wherein the pharmaceutical formulation is administered to the subject by an eye drop.

42. An eye drop for the treatment of an ocular neural disease, disorder or condition through topical application of said eye drop to the eye of a subject suffering from said disease, disorder or condition, comprising a therapeutically effective amount (5E,9E,13E) geranylgeranyl acetone and a solvent for said compound which is suitable for topical application to the eye of the subject, wherein (5E,9E,13E)geranylgeranyl acetone is present in a ratio of greater than 90:10 of (5E,9E,13E) to (5Z,9E,13E)geranylgeranyl acetone isomers.
